(19) United States
(12) Patent Application Publication
(10) Pub. No.: US 2025/0276987 A1
(43) Pub. Date: Sep. 4, 2025

(54) METHOD FOR MANUFACTURING DROMETRIZOLE TRISILOXANE (DMTS)

(71) Applicant: SERASOO CO., LTD, Cheonan-si (KR)

(72) Inventors: Dong Sun RYU, Seoul-si (KR); Jeong Ho SONG, Sejong-si (KR); Jun Hyeong KIM, Seoul-si (KR); Ye Rim HONG, Cheonan-si (KR); Yong Seok PARK, Pyeongtaek-si (KR); Ho Sang RYU, Seoul-si (KR)

(73) Assignee: SERASOO CO., LTD, Cheonan-si (KR)

(21) Appl. No.: 18/765,103

(22) Filed: Jul. 5, 2024

(30) Foreign Application Priority Data
Mar. 4, 2024 (KR) 10-2024-0030974

(51) Int. Cl. C07F 7/08; C07F 7/20

(52) U.S. Cl. CPC C07F 7/0889; C07F 7/20

(57) ABSTRACT

The present invention relates to a method for manufacturing drometrizole trisiloxane (DMTS), and more specifically, to a method for manufacturing drometrizole trisiloxane (DMTS) with high purity and yield in which solvents with high boiling points are not used, phosphates are used, any reaction auxiliaries are not used, the reaction time is very short, and impurities are low.

### TECHNICAL FIELD

The present invention relates to a method for manufacturing drometrizole trisiloxane (DMTS), and more specifically, to a method for manufacturing drometrizole trisiloxane (DMTS) with high purity and yield in which solvents with high boiling points are not used, phosphates are used, any reaction auxiliaries are not used, the reaction time is very short, and impurities are low.

### BACKGROUND ART

Sunscreens used for the purpose of preventing skin damage caused by ultraviolet rays are largely classified into chemical sunscreens and physical sunscreens.

Chemical sunscreens that chemically absorb ultraviolet rays organic include blockers such as cinnamic acid, salicylic acid, benzophenone, etc, while physical sunscreens that physically scatter and shield ultraviolet rays include inorganic blockers such as titanium dioxide, zinc oxide, etc.

Chemical sunscreens have the advantage of excellent UV blocking effect, but the wavelength range to which they are applied is narrow. When they are used in cosmetic compositions, they are sticky and shiny, and they can be absorbed into the skin in molecular form, causing skin irritation.

In the case of physical sunscreens, skin irritation is relatively lower than chemical sunscreens, but they have the disadvantage of causing a heavy feeling and bleaching.

Meanwhile, drometrizole trisiloxane (DMTS), known by the brand name Mexoryl XL or Silatrizole, is widely used as a sunscreen.

In relation to the production of DMTS, various studies are being conducted to produce high-purity compounds by effectively removing moisture and impurities.

However, the conventional technology has a long reaction time, a complicated reaction process, and a high content of impurities in the compound, so it cannot be stably used as a raw material for cosmetics, pharmaceuticals, and electronic devices.

(Patent Document 1) Korean Patent No. 10-2635085

(Patent Document 2) Korean Patent No. 10-2261129

(Patent Document 3) Korean Patent No. 10-0636953

### SUMMARY OF INVENTION

### Technical Problem

The present invention is intended to solve the problems of the prior art and provide a method for manufacturing DMTS with high purity and yield in which solvents with high boiling points are not used, phosphates are used, any reaction auxiliaries are not used, the reaction time is very short, and impurities are low.

### Solution to Problem

In order to achieve the above object, the present invention provides a method for manufacturing DMTS comprising the steps of
(a) mixing drometrizole, phosphate, and solvent to prepare a first mixture;
(b) adding methallyl halide to the first mixture and then reacting to produce a first product;
(c) adding distilled water to the first product and then phase separating to prepare a second mixture;
(d) distilling the second mixture to remove the solvent and then increasing the temperature to prepare methallyl drometrizole;
(e) preparing a third mixture by mixing the methallyl drometrizole and a solvent;
(f) cooling the third mixture, crystallizing, filtering and drying to obtain methallyl drometrizole;
(g) preparing a fourth mixture by mixing the methallyl drometrizole and a solvent;
(h) preparing a fifth mixture by mixing the fourth mixture and the catalyst solution;
(i) mixing the fifth mixture and solution and then reacting heptamethyltrisiloxane (HMTS) to produce a second product;
(j) distilling the second product to remove the solvent and then adding a solvent to prepare a sixth mixture; and
(k) cooling the sixth mixture, crystallizing, filtering and drying to obtain drometrizole trisiloxane (DMTS).

In one embodiment of the present invention, the phosphate in step (a) is potassium phosphate tribasic.

In one embodiment of the present invention, step (a) uses a mixture of ethyl acetate and dimethyl sulfoxide as a solvent.

In one embodiment of the present invention, step (d) is characterized in that methallyl drometrizole is produced by distilling the second mixture to remove the solvent and then reacting at 190 to 250° C. for 30 minutes to 5 hours.

In one embodiment of the present invention, step (e) is characterized by using a mixture of ethyl acetate and methanol as a solvent.

In one embodiment of the present invention, step (g) is characterized by using methyl ethyl ketone or acetone as a solvent.

### Advantageous Effects of Invention

The present invention can provide a method for manufacturing DMTS with high purity and yield in which solvents with high boiling points are not used, phosphates are used, any reaction auxiliaries are not used, the reaction time is very short, and impurities are low.

### DESCRIPTION OF EMBODIMENTS

The present invention will be described in detail below based on examples. The terms, examples, etc. used in the present invention are merely illustrative to explain the present invention in more detail and aid the understanding of those skilled in the art, and the scope of rights of the present invention should not be construed as limited thereto.

Technical terms and scientific terms used in the present invention, unless otherwise defined, represent meanings commonly understood by those skilled in the art in the technical field to which this invention pertains.

The present invention relates to a method for manufacturing DMTS comprising the steps of
(a) mixing drometrizole, phosphate, and solvent to prepare a first mixture;
(b) adding methallyl halide to the first mixture and then reacting to produce a first product;
(c) adding distilled water to the first product and then phase separating to prepare a second mixture;
(d) distilling the second mixture to remove the solvent and then increasing the temperature to prepare methallyl drometrizole;
(e) preparing a mixture by mixing the methallyl drometrizole and a solvent;
(f) cooling the third mixture, crystallizing, filtering and drying to obtain methallyl drometrizole;
(g) preparing a fourth mixture by mixing the methallyl drometrizole and a solvent;
(h) preparing a fifth mixture by mixing the fourth mixture and the catalyst solution;
(i) mixing the fifth mixture and heptamethyltrisiloxane (HMTS) solution and then reacting to produce a second product;
(j) distilling the second product to remove the solvent and then adding a solvent to prepare a sixth mixture; and
(k) cooling the sixth mixture, crystallizing, filtering and drying to obtain drometrizole trisiloxane (DMTS).

The present invention does not use a solvent with a high boiling point, uses phosphate, and does not use any reaction auxiliaries, and has a short reaction time and low impurities, making it possible to produce DMTS with high purity and yield.

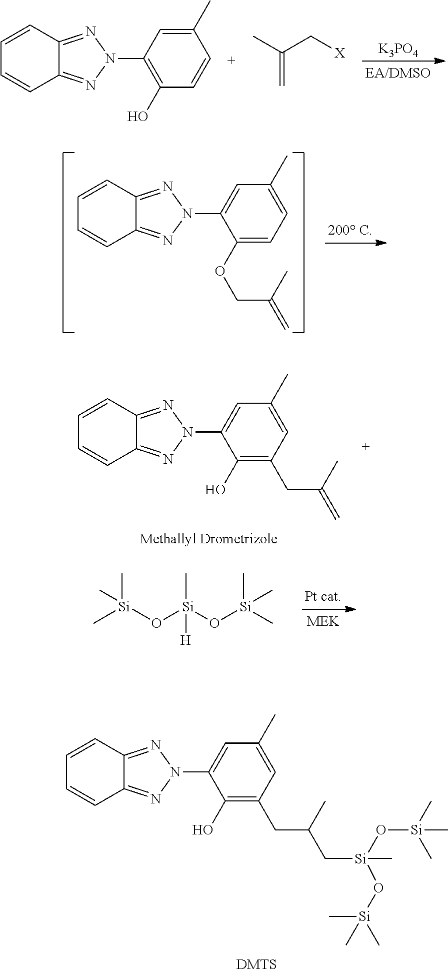

In step (a), the first mixture can be prepared by mixing drometrizole, phosphate, and solvent at 50 to 100° C. under a nitrogen atmosphere.

The first mixture may use 50 to 90 parts by weight of drometrizole and 60 to 90 parts by weight of phosphate based on 100 parts by weight of solvent. When the content satisfies the above numerical range, yield and purity can be maximized.

The phosphate includes potassium phosphate monobasic, potassium phosphate dibasic, potassium phosphate tribasic, sodium phosphate monobasic, sodium phosphate dibasic, sodium phosphate tribasic, lithium phosphate, calcium phosphate monobasic, calcium phosphate dibasic, calcium tribasic, magnesium phosphate monobasic, magnesium phosphate dibasic, magnesium phosphate tribasic, etc. and potassium phosphate tribasic is preferably used.

In addition, it is preferable to use 1 to 1.5 equivalents of phosphate per equivalent of drometrizole, and when the content satisfies the above numerical range, yield and purity can be maximized.

The solvent includes benzyl alcohol, ethanol, methanol, acetone, dimethyl sulfoxide, ethyl acetate, etc. In the present invention, a mixture of ethyl acetate and dimethyl sulfoxide can be used as a solvent, and the weight ratio of ethyl acetate and dimethyl sulfoxide is preferably 70 to 90:10 to 30. When the weight ratio satisfies the above numerical range, yield and purity can be maximized.

In step (b), a first product can be produced by adding methallyl halide to the first mixture and then reacting them.

At this time, the first product can be produced by adding the methallyl halide dropwise for 10 to 100 minutes and then reacting at 50 to 100° C. for 1 to 10 hours. The methallyl halide may be methallyl iodide, methallyl chloride, methallyl bromide, etc., and methallyl chloride is preferably used.

The content of the methallyl halide is preferably 20 to 60 parts by weight based on 100 parts by weight of the solvent.

In addition, it is preferable to use 1 to 1.5 equivalents of methallyl halide per equivalent of drometrizole, and when the content satisfies the above numerical range, yield and purity can be maximized.

In step (c), the second mixture can be prepared by adding distilled water to the first product and then phase separating it at 30 to 100° C.

The content of the distilled water is preferably 100 to 600 parts by weight based on 100 parts by weight of the solvent.

At this time, primary phase separation can be performed by adding 100 to 300 parts by weight of distilled water to the first product, and then secondary phase separation can be performed by adding 100 to 300 parts by weight of distilled water.

In step (d), methallyl drometrizole can be prepared by distilling the second mixture under normal pressure to remove the solvent, and then reacting the second mixture at 190 to 250° C. for 30 minutes to 5 hours.

In step (e), a third mixture can be prepared by mixing the methallyl drometrizole and the solvent at 60 to 100° C.

The solvent may be isopropyl alcohol, benzyl alcohol, ethanol, methanol, acetone, ethyl acetate, etc.

In the present invention, a mixture of ethyl acetate and methanol can be used as a solvent, and the weight ratio of ethyl acetate and methanol is preferably 20 to 50:50 to 80. When the weight ratio satisfies the above numerical range, yield and purity can be maximized.

Additionally, it is preferable to use 100 to 300 parts by weight of solvent based on 100 parts by weight of methallyl drometrizole.

In step (f), methallyl drometrizole can be obtained by cooling the third mixture, crystallizing it, and then filtering and drying it.

The third mixture produces crystals at 50 to 60° C. upon cooling, and can be cooled to room temperature and stirred, and then cooled to 0 to 5° C. and stirred for 1 to 5 hours.

The crystallized third mixture is filtered and separated into filtrate and solid content, and then the solid content is dried to obtain methallyl drometrizole.

In step (g), the fourth mixture can be prepared by mixing the methallyl drometrizole and the solvent at 20 to 60° C. under a nitrogen atmosphere.

The fourth mixture may use 100 to 300 parts by weight of methallyl drometrizole based on 100 parts by weight of the solvent. When the content satisfies the above numerical range, yield and purity can be maximized.

The solvent may be methyl ethyl ketone, isopropyl alcohol, acetone, toluene, ethyl acetate, etc.

In the present invention, it is preferable to use methyl ethyl ketone or acetone as a solvent.

In addition, a mixture of methyl ethyl ketone and acetone can be used as a solvent, and the weight ratio of methyl ethyl ketone and acetone is preferably 70 to 90:10 to 30. When the weight ratio satisfies the above numerical range, yield and purity can be maximized.

In step (h), the fifth mixture can be prepared by mixing the fourth mixture and the catalyst solution at 60 to 120° C.

The catalyst solution includes a catalyst and a solvent. In this case, the catalyst may be a Karstedt catalyst, platinum, etc.

The solvent may be methyl ethyl ketone, isopropyl alcohol, acetone, toluene, ethyl acetate, etc.

In the present invention, it is preferable to use methyl ethyl ketone or acetone as a solvent.

In addition, a mixture of methyl ethyl ketone and acetone can be used as a solvent, and the weight ratio of methyl ethyl ketone and acetone is preferably 70 to 90:10 to 30. When the weight ratio satisfies the above numerical range, yield and purity can be maximized.

The catalyst solution is preferably used in an amount of 0.1 to 5 parts by weight of catalyst per 100 parts by weight of solvent.

Additionally, it is preferable to use 5 to 30 parts by weight of the catalyst solution based on 100 parts by weight of the fourth mixture.

In step (i), the second product can be produced by mixing and reacting the fifth mixture and the heptamethyltrisiloxane (HMTS) solution.

At this time, the second product can be prepared by adding the heptamethyltrisiloxane solution dropwise for 10 to 30 minutes and then reacting at 60 to 120° C. for 1 to 10 hours.

The heptamethyltrisiloxane solution may include heptamethyltrisiloxane and a solvent.

The solvent may be methyl ethyl ketone, isopropyl alcohol, acetone, toluene, ethyl acetate, etc.

In the present invention, it is preferable to use methyl ethyl ketone or acetone as a solvent.

In addition, a mixture of methyl ethyl ketone and acetone can be used as a solvent, and the weight ratio of methyl ethyl ketone and acetone is preferably 70 to 90:10 to 30. When the weight ratio satisfies the above numerical range, yield and purity can be maximized.

The heptamethyltrisiloxane solution is preferably used at 300 to 1,000 parts by weight of heptamethyltrisiloxane based on 100 parts by weight of the solvent.

In addition, it is preferable to use 30 to 70 parts by weight of heptamethyltrisiloxane solution based on 100 parts by weight of the fifth mixture.

Additionally, it is preferable to use 1 to 1.5 equivalents of heptamethyltrisiloxane per 1 equivalent of methallyl drometrizole, and when the content satisfies the above numerical range, yield and purity can be maximized.

In step (j), the sixth mixture can be prepared by distilling the second product to remove the solvent, and then adding the solvent and mixing at 30 to 80° C. The solvent to be added may be isopropyl alcohol, benzyl alcohol, ethanol, methanol, acetone, ethyl acetate, etc.

The mixture of acetone and methanol can be used as a solvent, and the weight ratio of acetone and methanol is preferably 20 to 50:50 to 80. When the weight ratio satisfies the above numerical range, yield and purity can be maximized.

Additionally, it is preferable to use 200 to 600 parts by weight of solvent based on 100 parts by weight of methallyl drometrizole.

In step (k), the sixth mixture is cooled, crystallized, filtered, and dried to obtain drometrizole trisiloxane (DMTS).

The sixth mixture produces crystals at 25 to 30° C. upon cooling, and can be cooled to room temperature and stirred, and then cooled to 0 to 5° C. and stirred for 1 to 5 hours.

The crystallized sixth mixture is filtered and separated into filtrate and solid content, and then the solid content is dried to obtain drometrizole trisiloxane.

The present invention does not use solvents with high boiling points, uses phosphates, and does not use any reaction auxiliaries, and the reaction time is very short and impurities are small, making it possible to produce DMTS with high purity and yield.

The present invention will be described in detail below through examples. The following examples are merely illustrative for carrying out the present invention, and the content of the present invention is not limited by the following examples.

### Example 1

A first mixture was prepared by mixing 60 parts by weight of drometrizole, 70 parts by weight of potassium phosphate tribasic, 85 parts by weight of ethyl acetate, and 15 parts by weight of dimethyl sulfoxide at 65° C. under a nitrogen atmosphere.

A first product was prepared by adding 40 parts by weight of methallyl chloride dropwise to the first mixture for 30 minutes and then reacting them at 80° C. for 5 hours.

A second mixture was prepared by adding 250 parts by weight of distilled water to the first product and then phase separating at 50° C.

A methallyl drometrizole was prepared by distilling the second mixture under normal pressure to remove the solvent, and then reacting the second mixture at 200° C. for 2 hours.

A third mixture was prepared by mixing 100 parts by weight of methallyl drometrizole, 70 parts by weight of ethyl acetate, and 130 parts by weight of methanol at 70° C.

The third mixture was cooled to room temperature and stirred, and then cooled again to 5° C. and stirred for 2 hours to crystallize, and methallyl drometrizole was obtained through filtration and drying (pale yellow crystal; yield 94.5%).

A fourth mixture was prepared by mixing 150 parts by weight of methallyl drometrizole and 100 parts by weight of methyl ethyl ketone at room temperature under a nitrogen atmosphere.

A fifth mixture was prepared by mixing 100 parts by weight of the fourth mixture and 15 parts by weight of the catalyst solution at 75° C. At this time, the catalyst solution contained 1 part by weight of Karstedt catalyst (xylene solution containing 2% platinum) and 100 parts by weight of methyl ethyl ketone.

A second product was prepared by adding 50 parts by weight of heptamethyltrisiloxane (HMTS) solution dropwise to 100 parts by weight of the fifth mixture for 30 minutes, and then reacting at 75° C. for 5 hours. At this time, the heptamethyltrisiloxane solution contained 500 parts by weight of heptamethyltrisiloxane and 100 parts by weight of methyl ethyl ketone.

After distilling the second product to remove the solvent, acetone and methanol were added and mixed at 50° C. to prepare a sixth mixture. At this time, 105 parts by weight of acetone and 195 parts by weight of methanol were used based on 100 parts by weight of methallyl drometrizole.

The sixth mixture was cooled to room temperature and stirred, and then cooled again to 5° C. and stirred for 2 hours to crystallize. Drometrizole trisiloxane was obtained through filtration and drying (white crystal; yield 91.4%).

### Example 2

Drometrizole trisiloxane was obtained in the same manner as Example 1, except that 65 parts by weight of ethyl acetate and 35 parts by weight of dimethyl sulfoxide were used.

### Example 3

Drometrizole trisiloxane was obtained in the same manner as Example 1, except that 95 parts by weight of ethyl acetate and 5 parts by weight of dimethyl sulfoxide were used.

### Example 4

Drometrizole trisiloxane was obtained in the same manner as Example 1, except that 50 parts by weight of potassium phosphate tribasic was used.

### Example 5

Drometrizole trisiloxane was obtained in the same manner as Example 1, except that 100 parts by weight of potassium phosphate tribasic was used.

### Example 6

Drometrizole trisiloxane was obtained in the same manner as Example 1, except that 30 parts by weight of ethyl acetate and 170 parts by weight of methanol were used.

### Example 7

Drometrizole trisiloxane was obtained in the same manner as Example 1, except that 150 parts by weight of methallyl drometrizole and 100 parts by weight of acetone were mixed at room temperature under a nitrogen atmosphere to prepare a fourth mixture.

### Example 8

Drometrisol trisiloxane was obtained in the same manner as Example 1, except that 150 parts by weight of methallyl drometrizole, 80 parts by weight of methyl ethyl ketone, and 20 parts by weight of acetone were mixed at room temperature under a nitrogen atmosphere to prepare a fourth mixture.

### Comparative Example 1

Drometrizole trisiloxane was obtained in the same manner as Example 1, except that potassium carbonate (K₂CO₃) was used instead of potassium phosphate tribasic.

The yields of DMTS manufactured in the examples and comparative examples were measured and shown in Table 1.

TABLE 1

| | yield (%) |
|---|---|
| Example 1 | 91.4 |
| Example 2 | 86.2 |
| Example 3 | 85.8 |
| Example 4 | 84.7 |
| Example 5 | 85.0 |
| Example 6 | 85.9 |
| Example 7 | 91.0 |
| Example 8 | 91.7 |
| Comparative Example 1 | 81.3 |

As can be seen in Table 1, Examples 1 to 8 were found to have excellent yield. In particular, Examples 1, 7, and 8 have the best property.

On the other hand, it can be confirmed that the characteristics of Comparative Example 1 are inferior to those of the Examples.

1. A method for manufacturing DMTS comprising the steps of:
(a) mixing drometrizole, phosphate, and solvent to prepare a first mixture;
(b) adding methallyl halide to the first mixture and then reacting to produce a first product;
(c) adding distilled water to the first product and then phase separating to prepare a second mixture;
(d) distilling the second mixture to remove the solvent and then increasing the temperature to prepare methallyl drometrizole;
(e) preparing a third mixture by mixing the methallyl drometrizole and a solvent;
(f) cooling the third mixture, crystallizing, filtering and drying to obtain methallyl drometrizole;
(g) preparing a fourth mixture by mixing the methallyl drometrizole and a solvent;
(h) preparing a fifth mixture by mixing the fourth mixture and the catalyst solution;
(i) mixing the fifth mixture and heptamethyltrisiloxane (HMTS) solution and then reacting to produce a second product;
(j) distilling the second product to remove the solvent and then adding a solvent to prepare a sixth mixture; and
(k) cooling the sixth mixture, crystallizing, filtering and drying to obtain drometrizole trisiloxane (DMTS).

2. The method for manufacturing DMTS according to claim 1,
wherein the phosphate in step (a) is potassium phosphate tribasic.

3. The method for manufacturing DMTS according to claim 2,
wherein the solvent in step (a) is a mixture of ethyl acetate and dimethyl sulfoxide.

4. The method for manufacturing DMTS according to claim 3,
wherein in step (d), methallyl drometrizole is produced by distilling the second mixture to remove the solvent and then reacting at 190 to 250° C. for 30 minutes to 5 hours.

5. The method for manufacturing DMTS according to claim 4,
wherein the solvent in step (e) is a mixture of ethyl acetate and methanol.

6. The method for manufacturing DMTS according to claim 5,
wherein the solvent in step (g) is methyl ethyl ketone or acetone.
